(19) United States
(12) Patent Application Publication
(10) Pub. No.: US 2023/0059612 A1
(43) Pub. Date: Feb. 23, 2023

(54) System and Method for Urinary Blockage Removal

(71) Applicant: Julius J. Larry, III, Little Rock, AR (US)

(72) Inventor: Julius J. Larry, III, Little Rock, AR (US)

(21) Appl. No.: 17/409,786

(22) Filed: Aug. 23, 2021

(51) Int. Cl. A61B 17/32

(52) U.S. Cl. CPC A61B 17/32002; A61B 2017/00743

(57) ABSTRACT

The present invention relates to a urinary blockage removal system using a novel modified Foley catheter. The system is used for removing urinary bladder blockages of patients having obliterated prostatic urethra with dystrophic calcifications. In one exemplary embodiment, a 22-French modified Foley catheter is used as a sheath to maintain the patency of the lumen of the urethra. A tubular dental handpiece is operated by a dental lab motor and is advanced through the 22-French sheath to a point of the obstruction in the bladder. A dental bur can be pushed forward and drills through the calcification in the prostatic urethra and continues to the bladder neck. Using the dental bur, a blockage at the bladder neck can be removed.

### FIELD OF THE INVENTION

The present invention relates generally to urethra catheters. More specifically, the present invention relates to a system and method of removing blockage from a bladder using a 22-French (22 Fr) modified Foley catheter. The system and method are ideal for patients having an obliterated prostatic urethra with dystrophic calcifications by using the 22-French modified Foley catheter as a sheath. A dental bur can be used for drilling through the calcification in the prostatic urethra and then to continue to the bladder neck for removing the blockage. The prostatic urethra is the portion of the urethra that traverses the prostate. It originates in the region of the bladder neck, courses roughly 2.5 cm inferiorly and terminates at the membranous urethra. The prostatic urethra is surrounded by an inner circular layer and an outer longitudinal layer of smooth muscle. The dental bur can be pushed or inserted by attaching it to a tubular dental handpiece via a dental lab motor. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, urinary retention is a common problem in many individuals. Urinary retention is generally defined as a condition in which a person is not able to empty all the urine from the bladder. There are generally a lot of reasons for urinary retention in individuals. As an example, a person with a history of brachytherapy and radiation treatment for prostate cancer can succumb to urinary retention. Similarly, posterior urethral stenosis is a condition where the uppermost part of the urethra of a person narrows to the point that it prevents urine from passing out of the body.

When urine is not passed easily from the body, a person may face urinary tract infections, bloody urine, pain in the abdomen and the like. Further, urine retention can increase the pressure within the urinary tract and can adversely affect the kidneys, which in turn can lead to high blood pressure, leg swelling and further kidney damage.

Usually, a Foley catheter is used for removing urine from the bladder in cases of urinary retention. A Foley catheter is a flexible tube that a clinician passes through the urethra and into the bladder to drain urine. It is the most common type of indwelling urinary catheter. The Foley catheter can include two tubes or lumens. The tubes or lumens are separated channels running down the length of the catheter. One lumen, open at both ends, can drain urine into a collection bag, and the other lumen can have a valve on the outside end and connects to a balloon at the inside tip. The balloon can be inflated with sterile water when it lies inside the bladder to stop the catheter from slipping out. Manufacturers typically produce Foley catheters using silicone or coated natural latex. In some cases, urethra catheters are used for draining urine in which a catheter is inserted into a urethra opening. The balloon is inflated slowly using sterile water to the recommended volume on the catheter for enabling flow of urine.

In cases where the urethra catheter cannot be inserted into the urethra, a suprapubic catheter can be used. The suprapubic catheter is a hollow flexible tube for draining urine from the bladder. The suprapubic catheter is inserted into the bladder through a cut or incision in the stomach, a few inches below the navel. In many cases, intermittent and temporary urinary catheters are used. These catheters are inserted several times a day, for just long enough to drain the bladder, and are removed thereafter.

Further, to diagnose conditions such as Prostatic Urethral Stenosis, a conventional 21-French rigid scope can be inserted into the urethra to view and detect an obliterated urethra and dystrophic calcifications. As known in the art, dystrophic calcifications comprise the calcification occurring in degenerated or necrotic tissue, as in hyalinized scars, degenerated foci in leiomyomas and caseous nodules. This occurs as a reaction to tissue damage, including as a consequence of medical device implantation. Dystrophic calcification occurs as a result of chronic inflammation or tissue necrosis. It is associated with several medical conditions such as collagen vascular disease, scleroderma and systemic lupus erythematosus, as well as with soft tissue injuries from trauma. An obliterated urethra is a trauma to the urethra, or a condition that can result after treatment for prostate cancer. While using the suprapubic catheter, methylene blue may be inserted into the suprapubic catheter. However, due to an obliterated prostatic urethra with dystrophic calcifications, blue dye leakage may not be visualized from the bladder. Methylene blue can be used to treat methemoglobinemia and urinary tract infections. Methylene blue can also be used as a dye or staining agent to make certain body fluids and tissues easier to view during surgery or on an x-ray or other diagnostic exam.

Therefore, there exists a long felt need in the art for a urinary retention diagnosis system and method that effectively removes blockage at the bladder neck. There is also a long felt need in the art for a urinary retention diagnosis system and method that maintains patency of the lumen of the urethra. Additionally, there is a long felt need in the art for a urinary retention diagnosis system and method that uses conventional suprapubic catheters. There is a long felt need in the art for a urinary retention diagnosis system and method that uses a modified Foley catheter. Moreover, there is a long felt need in the art for a urinary retention diagnosis system and method that uses methylene blue for checking the unblocking of the bladder. Finally, there is a long felt need in the art for a urinary retention diagnosis system and method that removes blockage from the bladder neck and obliterated prostatic urethra.

The subject matter disclosed and claimed herein, in one embodiment thereof, comprises a novel urinary retention diagnosis system for a bladder neck. The bladder neck is a group of muscles that connect the bladder to the urethra. The muscles tighten to hold urine in the bladder, and relax to release it through the urethra. Urinary problems occur when abnormalities block the bladder neck, and prevent it from opening completely during urination. More specifically, the urinary retention diagnosis system includes: a 22-French (22 Fr) modified Foley catheter designed to work as a sheath: a flexible and tubular dental handpiece; the flexible and tubular dental handpiece can be operated by a dental lab motor in order to reach a point of the obstruction in the bladder through the sheath; a knob on the dental lab motor for turning the flexible and tubular dental handpiece at a low speed at the point of obstruction; and a spear-shaped dental bur for drilling through the calcification in the prostatic urethra to the bladder neck.

In this manner, the novel urinary retention diagnosis system of the present invention accomplishes all of the forgoing objectives, and provides a relatively safe, easy, and convenient solution to allow surgeons to remove a blockage from the bladder neck where the calcification is present in the prostatic urethra. The system is easy to use and can be operated using a dental lab motor.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a urinary retention diagnosis system for removing a blockage from a bladder neck. The system is configured to work with patients having obliterated prostatic urethra with dystrophic calcifications. More specifically, the urinary retention diagnosis system includes a 22-French modified Foley catheter designed to work as a sheath, a flexible and tubular dental handpiece, the flexible and tubular dental handpiece can be operated by a dental lab motor in order to reach the point of the obstruction in the bladder through the sheath, a knob on the dental lab motor for turning the flexible and tubular dental handpiece at a low speed at the point of obstruction and a spear-shaped dental bur for drilling through the calcification in the prostatic urethra to the bladder neck for removing the blockage.

In a further embodiment of the present invention, a method for removing a blockage from a bladder neck of a patient with obliterated prostatic urethra with dystrophic calcifications is described. The method includes: initially anesthetizing the patient with a lumbar block and twilight sedation; placing the patient in a dorsal lithotomy position, prepped and draped; inserting a 22-French modified Foley catheter to act as a sheath to maintain the patency of the lumen of the urethra; advancing tubular dental handpiece using a dental lab motor through the sheath to the point of obstruction in the bladder; turning the tubular dental handpiece at low speed using a control knob on the dental lab motor; drilling a spear-shaped dental bur through the calcification in the prostatic urethra to the bladder neck; removing the blockage; placing a wire through a 22-French catheter into the bladder; inflating a balloon; and removing the 22-French catheter.

In a further embodiment of the present invention, the 22-French modified Foley catheter is inserted to the point of the obstruction to act as a sheath to maintain the patency of the lumen of the urethra.

In yet a further embodiment of the present invention, the 22-French modified Foley catheter is a thin and hollow sterile tube through which a tubular dental handpiece can be inserted into the bladder to drain urine.

In yet a further embodiment of the present invention, the system and method are used for patients having dystrophic calcification of the prostate after cryotherapy. Methylene blue can also flow through the newly-opened bladder neck and obliterated prostatic urethra, using the method and the system described supra.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a 22-French modified Foley catheter of the present invention in accordance with the disclosed architecture;

FIG. 2 illustrates a perspective view showing a dental lab motor used in a urinary retention diagnosis system of the present invention for removing a blockage from a bladder neck in accordance with the disclosed architecture;

FIG. 3 illustrates a perspective view showing various shapes of a dental bur that can be used in the present invention in accordance with the disclosed architecture;

FIG. 4 illustrates a perspective view showing operation of removal of a blockage from bladder neck of a patient in accordance with the disclosed architecture;

FIG. 5 illustrates a block diagram showing components of the urinary blockage removal system of the present invention in accordance with the disclosed architecture; and

FIG. 6 illustrates a flow diagram showing steps followed in the urinary blockage removal system for removing a blockage from bladder neck in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a urinary retention diagnosis system and method that effectively removes a blockage at the bladder neck. There is also a long felt need in the art for a urinary retention diagnosis system and method that maintains patency of the lumen of the urethra. Additionally, there is a long felt need in the art for a urinary retention diagnosis system and method that uses conventional suprapubic catheters. There is a long felt need in the art for a urinary retention diagnosis system and method that uses a modified Foley catheter. Moreover, there is a long felt need in the art for a urinary retention diagnosis system and method that uses methylene blue for checking the unblocking of the bladder. Finally, there is a long felt need in the art for a urinary retention diagnosis system and method that removes a blockage from the bladder neck and obliterated prostatic urethra.

The present invention, in one exemplary embodiment, is a method for removing a blockage from the bladder neck of a patient with obliterated prostatic urethra comprising dystrophic calcifications. The method includes: initially anesthetizing the patient with a lumbar block and twilight sedation; placing the patient in a dorsal lithotomy position, prepped and draped; inserting a 22-French modified Foley catheter to act as a sheath to maintain the patency of the lumen of the urethra; advancing a tubular dental handpiece using a dental lab motor through the sheath to a point of obstruction in the bladder; turning the tubular dental handpiece at low speed using a control knob on the dental lab motor; drilling a spear-shaped dental bur through the calcification in the prostatic urethra to the bladder neck; removing the blockage; placing a wire through a 22-French catheter into the bladder; inflating a balloon; and removing the 22-French catheter.

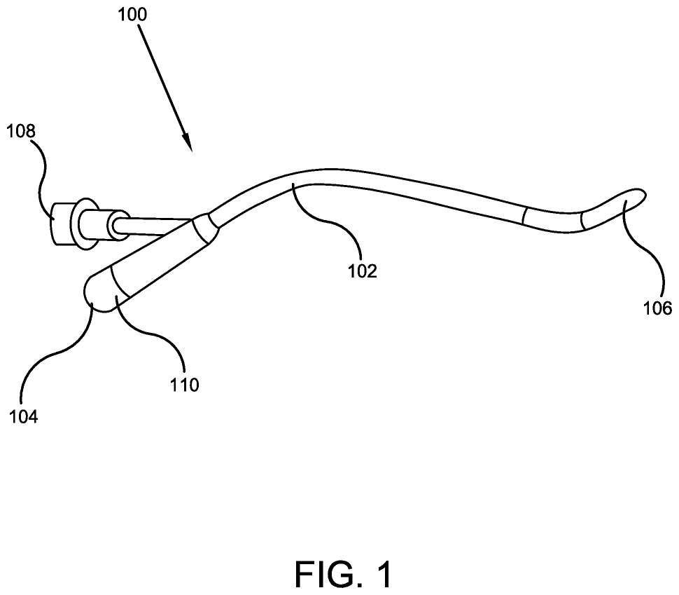

Referring initially to the drawings, FIG. 1 illustrates a 22-French modified Foley catheter of the present invention in accordance with the disclosed architecture. The 22-French modified Foley catheter is generally designated with the reference numeral 100. The modified catheter 100 includes a catheter body 102 with a proximal end 104 and a distal end 106. The catheter also includes an inflation port 108 and a drainage port 110. The gap between the two walls is connected via an inflation lumen to the inflation port 110. The modified catheter 100 includes a hollow tube that acts as a sheath or cover for maintaining patency of the lumen within the urethra. The hollow catheter 100 enables a flexible and tubular dental handpiece operated by a dental lab motor to a point of the obstruction in a bladder neck. By way of a non-limiting example, the outer wall of the modified catheter 100 can be of a highly flexible biocompatible silicone elastomer whilst the inner wall may be comprised of a stiffer silicone elastomer.

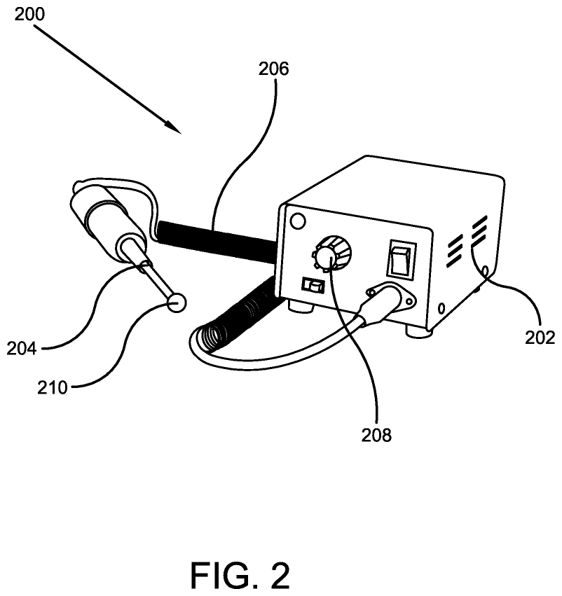

FIG. 2 illustrates a perspective view showing a dental lab motor used in the urinary retention diagnosis system of the present invention for removing a blockage from a bladder neck in accordance with the disclosed architecture. For removing a blockage from the neck of a bladder of a patient having obliterated prostatic urethra with dystrophic calcifications, a dental lab motor 200 can be used. The dental lab motor 200 can be commercially available with the modified catheter 100 of FIG. 1. The dental lab motor 200 can be compact in size and include a torque of 2.9 Ncm. The motor 200 includes a micrometer engine 202. A flexible and tubular dental handpiece 204, also referred to as “snake” in the disclosure, can be connected to the motor 200 through a flexible pipe 206. The handpiece 204 can be operated and advanced through the 22-French modified catheter sheath to the point of the obstruction in the bladder of a patient. In one potential embodiment, the flexible and tubular dental handpiece 204 can be up to 24″ in length. At the point of the obstruction in the bladder of a patient, the handpiece 204 can be turned on at low-speed using a control knob 208 on the dental lab motor 200.

A “spear-shaped” dental bur 210 can be attached to the handpiece 204 and pushed forward to drill through the calcification in the prostatic urethra, and continue onward to the bladder neck of the patient. Using the dental bur 210, the blockage at the bladder neck can be removed and thereafter the handpiece or “snake” 204 can be withdrawn.

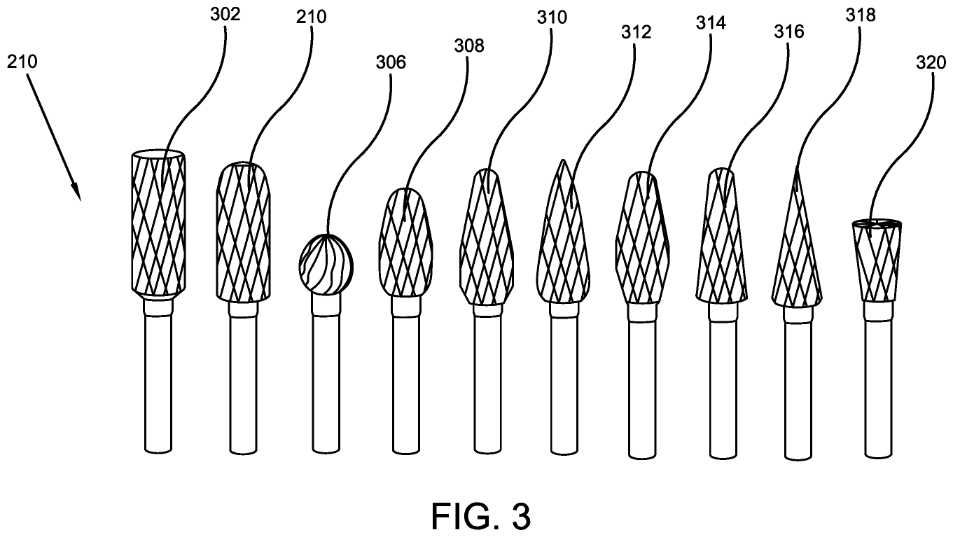

FIG. 3 illustrates a perspective view showing various shapes of a dental bur used in the present invention in accordance with the disclosed architecture. The dental bur 210 used in the present invention can be of various shapes, and may be made of a soft material or a hard material such as carbide. The dental bur 210 includes a broad and fine crosscut for smooth removal of the blockage from neck of the bladder. The spear-shape is advantageous since the shape is useful for cavity preparation, endo access points and restoration removal. The dental bur 210 can include a flat cylindrical 302, cylindrical 304, round 306, slightly tapered 308, tapered 310, slightly conical 312, convex 314, frustrum 316, conical 318 or shortened cylindrical 320 tip profile or shape. A particular profile or shape can be chosen based on the shape and size of the neck of the bladder of the patient.

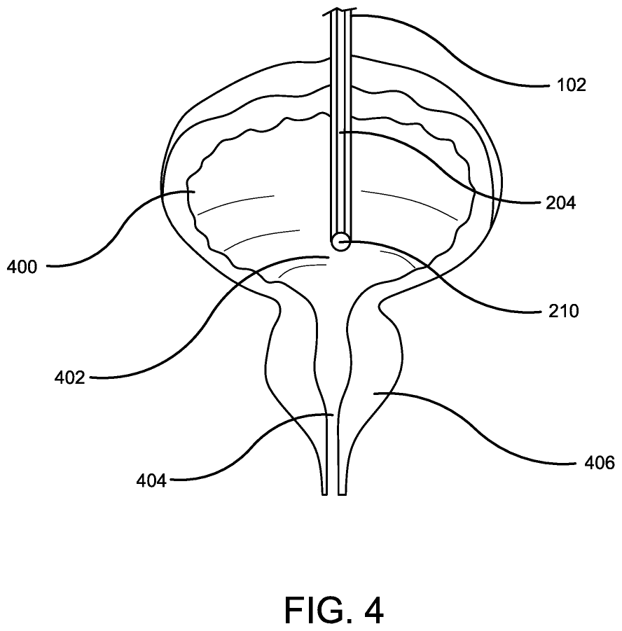

FIG. 4 illustrates a perspective view showing the operation of the removal of a blockage from the bladder neck of a patient, in accordance with the disclosed architecture. It should be appreciated that the present invention involves the removal of a blockage from the bladder neck 402 using a modified Foley catheter used as a sheath 100. The sheath 100 is used as a medium for the “snake” 204 including the dental bur 210 attached to remove the blockage from the neck 402 of the bladder 400, so that urine can be passed through the urethra 404. It should be appreciated that any patient having obliterated prostatic urethra with dystrophic calcifications may have a urethra blockage. The prostate gland 406, after treatment of prostate cancer, may include calcifications. After the bladder neck 402 is opened, methylene blue (i.e. dye) can be installed in a suprapubic catheter, which can then flow easily through the opened bladder neck 402. After the flowing of methylene blue, a wire can be placed in the bladder neck 402, and then the modified 22-French catheter 100 can be placed over the wire into the bladder 400 and a balloon (not shown) can subsequently be inflated. Bladder neck contracture can also be treated using the present invention where the bladder neck is narrow due to radiation therapy for prostate cancer.

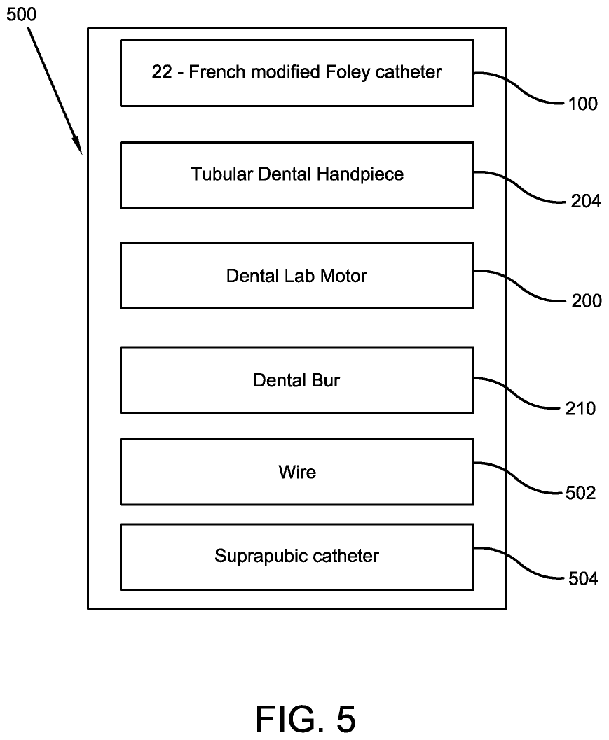

FIG. 5 illustrates a block diagram showing components of the urinary blockage removal system of the present invention in accordance with the disclosed architecture. The urinary blockage removal system 500 includes the 22-French modified Foley catheter 100 used as a sheath to maintain the patency of the lumen of the urethra. The tubular dental handpiece 204 is operated by the dental lab motor 200 and is advanced through the 22-French sheath 100 to a point of the obstruction in the bladder. The tubular dental handpiece 204 can be twenty-four inches in length. The dental bur 210 can be a “spear-shaped” bur, or similar, and can be pushed forward. The dental bur 210 drills through the calcification in the prostatic urethra and continues to the bladder neck. Using the dental bur 210, the blockage at the bladder neck is removed. A wire 502 can be placed in the bladder and then the 22-French catheter 100 can be placed over the wire 502 into the bladder, wherein a balloon can subsequently be inflated. The suprapubic catheter 504 inserted through the stomach can be removed to complete the procedure.

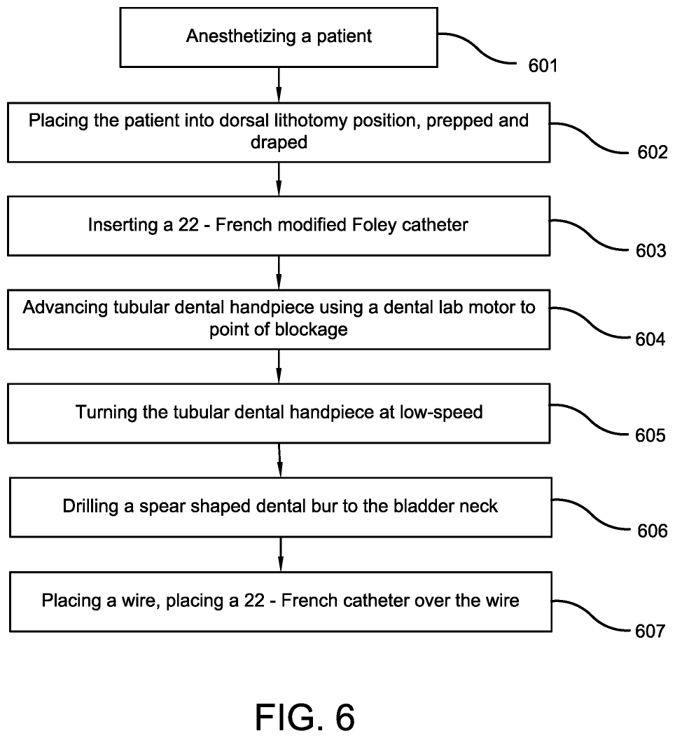

FIG. 6 illustrates a flow diagram showing steps followed in the urinary blockage removal system for removing a blockage from the bladder neck in accordance with the disclosed architecture. Initially in step 601, a patient is anesthetized with a lumbar block and twilight sedation. In step 602, the patient is placed in a dorsal lithotomy position, prepped, and draped. Then, in step 603, a 22-French modified Foley catheter is inserted into the bladder to act as a sheath to maintain the patency of the lumen of the urethra. In step 604, a tubular dental handpiece is advanced using a dental lab motor through the sheath to a point of obstruction in the bladder. In step 605, the tubular dental handpiece is turned at a low speed using a control knob on the dental lab motor and then in step 606, a spear-shaped dental bur is drilled through the calcification in the prostatic urethra to the bladder neck to remove the blockage. Finally in step 607, a wire is placed and then a 22-French catheter is placed over the wire into the bladder for inflating a balloon. Subsequent to inflating the balloon, the 22-French catheter is removed.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “urinary retention diagnosis system”, “urinary blockage removal system”, “system”, and “urinary retention diagnosis system” are interchangeable and refer to the urinary blockage removal system 500 of the present invention. Similarly, as used herein “22-French modified Foley catheter”, “modified catheter”, and “hollow catheter” are interchangeable and refer to the 22-French (22 Fr) modified Foley catheter 100 of the present invention.

Notwithstanding the forgoing, the 22-French modified Foley catheter 100 and the urinary blockage removal system 500 of the present invention can be of any suitable size and configuration as is known in the art without affecting the overall concept of the invention, provided that it accomplishes the above-stated objectives. One of ordinary skill in the art will appreciate that the size, configuration and material of the 22-French modified Foley catheter 100 and the urinary blockage removal system 500 as shown in the FIGS. are for illustrative purposes only, and that many other sizes and shapes of the 22-French modified Foley catheter 100 and the urinary blockage removal system 500 are well within the scope of the present disclosure. Although the dimensions of the 22-French modified Foley catheter 100 and the urinary blockage removal system 500 are important design parameters for user convenience, the 22-French modified Foley catheter 100 and the urinary blockage removal system 500 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for removing an obstruction from a bladder neck of a urethra of a patient, the method comprising the steps of:
anesthetizing the patient with at least twilight sedation;
inserting a catheter to act as a sheath to maintain the patency of a lumen of the urethra;
mounting a dental bur to a tubular dental handpiece;
advancing said tubular dental handpiece using a dental lab motor through said catheter to a point of obstruction in the urethra;
turning said dental bur at low-speed using a control knob on said dental lab motor;
drilling said dental bur through said obstruction in the urethra; and
removing said obstruction.

2. The method of claim 1, wherein said catheter is a modified Foley 22-French catheter.

3. The method of claim 2, wherein said urethra is a prostatic urethra.

4. The method of claim 3, wherein said obstruction is an obliterated prostatic urethra comprising dystrophic calcifications.

5. The method of claim 1 further comprising a step of placing the patient in a dorsal lithotomy position.

6. The method of claim 1, wherein a profile of said dental bur is a spear shape.

7. The method of claim 3, wherein said obstruction is calcification in said prostatic urethra.

8. The method of claim 1, wherein said dental bur includes a profile selected from the group consisting of a generally flat cylindrical shape, a cylindrical shape, a round shape, a slightly tapered shape, a tapered shape, a slightly conical shape, a convex shape, a frustrum shape, a conical shape, and a shortened cylindrical shape.

9. A catheter system for removing blockage from a bladder neck of a prostatic urethra of a patient, the catheter system comprising:
a catheter including a catheter body having a proximal end and a distal end, wherein said catheter includes a hollow tube for maintaining patency of a lumen within the prostatic urethra;
a flexible and tubular dental handpiece extending through said hollow tube to a point of the blockage in the bladder neck;
a dental bur mounted to an end of said dental handpiece; and
a dental lab motor for turning said dental bur at said distal end of said catheter, wherein said dental bur drills through said blockage in the bladder neck.

10. The catheter system for removing blockage from a bladder neck of claim 9, wherein said catheter includes an inflation port and a drainage port.

11. The catheter system for removing blockage from a bladder neck of claim 9 further comprising placing a wire through said catheter into the bladder and inflating a balloon at said distal end of said catheter.

12. The catheter system for removing blockage from a bladder neck of claim 9, wherein said blockage is an obliterated prostatic urethra comprising dystrophic calcifications.

13. The catheter system for removing blockage from a bladder neck of claim 9, wherein a profile of said dental bur is a spear shape.

14. The catheter system for removing blockage from a bladder neck of claim 9, wherein said obstruction is calcification in the prostatic urethra.

15. The catheter system for removing blockage from a bladder neck of claim 9, wherein said dental bur includes a profile selected from the group consisting of generally a flat cylindrical shape, a cylindrical shape, a round shape, a slightly tapered shape, a tapered shape, a slightly conical shape, a convex shape, a frustrum shape, a conical shape, and a shortened cylindrical shape.

16. The catheter system for removing blockage from a bladder neck of claim 9, wherein said catheter is a modified Foley 22-French catheter.

17. The catheter system for removing blockage from a bladder neck of claim 9, wherein said catheter includes an outer wall of a material of flexible biocompatible silicone elastomer.

18. The catheter system for removing blockage from a bladder neck of claim 17, said catheter includes an inner wall of a material of silicone elastomer.

19. The catheter system for removing blockage from a bladder neck of claim 9, wherein said dental lab motor includes a torque of 2.9 Ncm.

20. A method for removing an obstruction from a bladder neck of a urethra of a patient, the method comprising the steps of:
anesthetizing the patient with at least twilight sedation;
inserting a catheter to act as a sheath to maintain the patency of a lumen of the urethra, wherein said urethra is a prostatic urethra;
mounting a dental bur to a tubular dental handpiece;
advancing said tubular dental handpiece using a dental lab motor through said catheter to a point of obstruction in said prostatic urethra, wherein said obstruction is an obliterated prostatic urethra comprising dystrophic calcifications;
turning said dental bur at low-speed using a control knob on said dental lab motor;
drilling said dental bur through said obstruction in said prostatic urethra; and
removing said obstruction.
